(19) United States
(12) Patent Application Publication
(10) Pub. No.: US 2024/0398677 A1
(43) Pub. Date: Dec. 5, 2024

(54) ANTIPERSPIRANT COMPOSITIONS

(71) Applicant: ELEMENTIS SPECIALTIES, INC., East Windsor, NJ (US)

(72) Inventors: Rey ORDIALES, Kendall Park, NJ (US); Victoria CARDARELLI, Hamilton, NJ (US); Barbara M. SCHMIDT, Metuchen, NJ (US); Kimberly BURCH, Chesterfield, NJ (US)

(73) Assignee: ELEMENTIS SPECIALTIES, INC., East Windsor, NJ (US)

(21) Appl. No.: 18/203,953

(22) Filed: May 31, 2023

(51) Int. Cl. A61K 8/36; A61K 8/37; A61K 8/49; A61K 8/9789; A61Q 15/00

(52) U.S. Cl. CPC A61K 8/36; A61K 8/37; A61K 8/498; A61K 8/9789; A61Q 15/00; A61K 2800/48; A61K 2800/874

(57) ABSTRACT

An antiperspirant composition comprising an antiperspirant agent is described. The antiperspirant agent comprises an optionally substituted benzenepropenoic acid, a flavonoid, an ester of gallic acid, ellagic acid or a mixture of two or more thereof. Methods and uses of the antiperspirant composition and the antiperspirant agent are also described.

### FIELD OF THE INVENTION

The invention relates to an antiperspirant composition. The invention also relates to a method of reducing or preventing perspiration and to the use of certain naturally obtainable ingredients, particularly phytochemicals, as antiperspirant agents.

### BACKGROUND OF THE INVENTION

Antiperspirant compositions are a well-known type of product in the field of personal care and are used to eliminate or reduce body odour, which is important for daily hygiene. These compositions are available in a variety of forms for administration, such as solid sticks, roll-ons, wipes, aerosol sprays and pump sprays.

The compositions generally contain an active ingredient, often referred to as an antiperspirant agent, which may narrow or block the ducts of sweat glands to reduce or prevent perspiration. The antiperspirant agent may also provide a deodorizing effect if it is able to mask odors or it may have antimicrobial activity.

Antiperspirant agents in common use are generally aluminum or aluminum-zirconium salts, such as aluminum chlorohydrate or aluminum zirconium tetrachlorohydrex gly (AZG). These salts dissolve on the skin and plug pores to reduce or prevent sweating.

The search for alternative compounds with similar functionalities have been ongoing to match or improve the sweat reduction properties of aluminum or aluminum zirconium salts.

### SUMMARY OF THE INVENTION

In a first aspect, the invention provides an antiperspirant composition. The antiperspirant composition comprises an antiperspirant agent, wherein the antiperspirant agent comprises an optionally substituted benzenepropenoic acid, a flavonoid, an ester of gallic acid, ellagic acid or a mixture of two or more thereof. The optionally substituted benzenepropenoic acid, the flavonoid, the ester of gallic acid or the ellagic acid may be phytochemicals.

The first aspect of the invention also provides an antiperspirant composition comprising an antiperspirant agent, wherein the antiperspirant agent comprises a phytochemical selected from a flavonoid, an optionally substituted benzenepropenoic acid, ellagic acid, an ester of gallic acid and a mixture of two or more thereof.

In a second aspect, the invention also provides an antiperspirant composition. The antiperspirant composition comprises either: (a) a natural extract selected from cinnamon extract, black ginger extract, green tea extract, saberry extract, and a mixture of two or more thereof; or (b) an antiperspirant agent obtained or obtainable from a natural extract, wherein the natural extract is selected from cinnamon extract, black ginger extract, green tea extract, saberry extract and a mixture of two or more thereof. The antiperspirant agent in (b) may be present in the antiperspirant composition in the form of the natural extract in (a).

It has surprisingly been found that certain classes of phytochemicals or plant extracts possess antiperspirant properties. These naturally derivable materials can be used as antiperspirant agents, particularly in personal care products. They can provide a comparable antiperspirant effect to the aluminum or aluminum-zirconium salts that are typically included in most commercially available antiperspirant products. Thus, the phytochemicals or plant extracts can be included as antiperspirant agents instead of the aluminum or aluminum-zirconium salts. These materials, particularly the natural extracts, may also impart a pleasant odor to the antiperspirant composition. There is also no concern over risks to health associated with these ingredients.

A third aspect of the invention relates to a method of reducing or preventing perspiration. The method comprises applying an antiperspirant composition in accordance with the first or second aspects of the invention to skin.

The third aspect of the invention also includes the use of the antiperspirant composition to reduce or prevent perspiration.

A fourth aspect of the invention relates to the use of an optionally substituted benzenepropenoic acid, a flavonoid, ellagic acid, an ester of gallic acid or a mixture of two or more thereof as an antiperspirant agent for reducing or preventing perspiration.

The fourth aspect also includes the use of a phytochemical as an antiperspirant agent for reducing or preventing perspiration, wherein the phytochemical is an optionally substituted benzenepropenoic acid, a flavonoid, an ester of gallic acid, ellagic acid or a mixture of two or more thereof.

A fifth aspect of the invention relates to the use of a natural extract as an antiperspirant agent for reducing or preventing perspiration, wherein the natural extract is selected from cinnamon extract, black ginger extract, green tea extract, saberry extract and a mixture of two or more thereof.

### DETAILED DESCRIPTION OF THE INVENTION

The following definitions apply to the disclosure herein, including all aspects of the invention and their embodiments.

The term “personal care” as used herein, whether used alone or in conjunction with another term, refers to a non-therapeutic use, such as a non-therapeutic, cosmetic use. This term typically refers to use for personal hygiene.

The compounds and intermediates described herein may be named according to the IUPAC (International Union for Pure and Applied Chemistry) or the CAS (Chemical Abstracts Service) nomenclature systems or by their commercial trade names. It should also be understood that any reference to a compound described herein, such as a polyurea, a primary monoamine, a diamine or a triamine, encompasses all stereoisomers (e.g. cis and trans isomers) and/or optical isomers (e.g. R and S enantiomers) of such compounds, in substantially pure form and/or any mixtures of the foregoing in any ratio.

The term “phytochemical” as used herein refers to a chemical compound that is produced by a plant (e.g. through primary or secondary metabolism). In other words, a phytochemical is a naturally occurring, biologically active compound found in a plant. The phytochemical typically has biological activity that protects the plant from the environment, disease or a predator.

The term “flavonoid” as used herein refers to a compound having a 15-carbon core structure (e.g. skeleton) consisting of two phenyl rings (referred to as the “A” and “B” rings) and a heterocyclic ring (referred to as the “C” ring), which contains an oxygen atom. The term “flavonoid” is used in the conventional, restricted sense as referring only to those compounds with a C₆—C₃—C₆ carbon framework (e.g. core structure or skeleton) having the structure of a chromane or a 1-benzopyran (chromene), in which the fused benzene ring is designated as ring A and the 3,4-dihydro-2H-pyran or the pyran as ring C, along with a phenyl group (ring B) on ring C (see, for example, the “Nomenclature of flavonoids” by A.P. Rauter et al.—Reference 1). The general structure of a flavonoid with ring labelling is shown below.

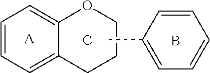

Depending on the position of the linkage of ring B to the chromane/1-benzopyran (chromene) moiety, the term “flavonoid” comprises, or consists of, the sub-classes of compounds known as 2-flavonoids, 3-flavonoids (e.g. also known as isoflavonoids) and 4-flavonoids (e.g. also known as neoflavonoids).

The term “2-flavonoid” as used herein refers to a compound having a core structure or skeleton as represented by formula (A-1).

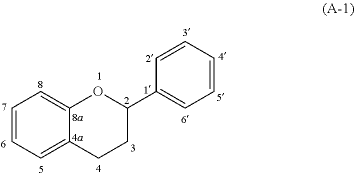 [SMILES: c1ccc(C2CCc3ccccc3O2)cc1]

In formula (A-1), the atoms of the core structure are numbered in accordance with the IUPAC numbering scheme for this sub-class of compounds.

For the avoidance of doubt, formula (A-1) represents the core structure or skeleton of a 2-flavonoid. Rings A to C of the core structure may include substituents. Ring C may include a carbon-carbon double bond (in addition to the unsaturation arising from the fused phenyl group, which is ring A) and/or a carbonyl group.

In general, the term “2-flavonoid” includes a flavan, a flavone or a flavanol.

When ring C in formula (A-1) does not include a carbon-carbon double bond or a carbonyl group, then formula (A-1) represents a flavan. The term “flavan” refers to a compound with a 2-phenyl-3,4-dihydro-2H-1-benzopyran skeleton.

When ring C in formula (A-1) is substituted with a hydroxy group, such as at the 3-position and/or the 4-position of ring C, then the 2-flavonoid is a flavanol.

The core structure or skeleton of the flavanol may be a flavan-3-ol (as represented by formula (A-2)), a flavan-4-ol (as represented by formula (A-3)) or a flavan-3,4-diol (as represented by formula (A-4)).

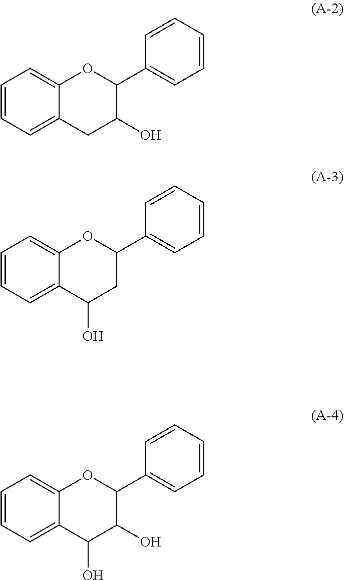 [SMILES: OC1Cc2ccccc2OC1c1ccccc1 ; OC1CC(c2ccccc2)Oc2ccccc21 ; OC1c2ccccc2OC(c2ccccc2)C1O]

When ring C in formula (A-1) includes a carbonyl group, such that the compound is derived from a 2-phenyl-2,3-dihydro-4H-1-benzopyran-4-one skeleton, then the 2-flavonoid is a flavanone, specifically a flavan-4-one (e.g. as represented by formula (A-5)).

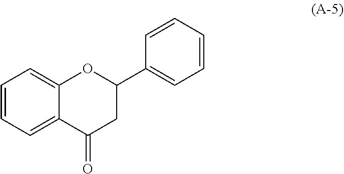 [SMILES: O=C1CC(c2ccccc2)Oc2ccccc21]

It is preferred that the core structure or skeleton of the 2-flavonoid is not an anthocyanidin (also known as an anthocyanin). An anthocyanidin is a compound derived from a flavylium (2-phenyl-1λ⁴-benzopyran-1-ylium or 2-phenylchromenylium) ion.

The term “3-flavonoid” or “isoflavonoid” as used herein refers to a compound having a core structure or skeleton as represented by formula (B-1).

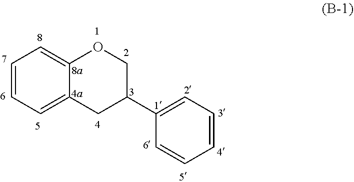 [SMILES: c1ccc(C2COc3ccccc3C2)cc1]

In formula (B-1), the atoms of the core structure are numbered in accordance with the IUPAC numbering scheme for this sub-class of compounds.

For the avoidance of doubt, formula (B-1) represents the core structure or skeleton of a 3-flavonoid. Rings A to C of the core structure may include substituents. Ring C may include a carbon-carbon double bond (in addition to the unsaturation arising from the fused phenyl group, which is ring A) and/or a carbonyl group.

In general, the term “3-flavonoid” or “isoflavonoid” includes isoflavans and isoflavones.

When ring C in formula (B-1) does not include a carbon-carbon double or a carbonyl group, then formula (B-1) represents an isoflavan. The term “isoflavan” refers to a compound with a 3-phenyl-3,4-dihydro-2H-1-benzopyran skeleton.

When ring C in formula (B-1) includes a carbonyl group, such as, for example, when the compound is derived from a 3-phenyl-4H-1-benzopyran-4-one (3-phenyl-4H-chromen-4-one) skeleton, then the 3-flavonoid is an isoflavanone (e.g. as represented by formula (B-2)).

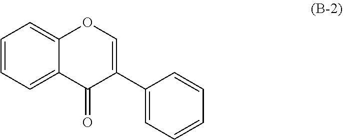 [SMILES: O=c1c(-c2ccccc2)coc2ccccc12]

The term “4-flavonoid” or “neoflavonoid” as used herein refers to a compound having a core structure or skeleton as represented by formula (C-1).

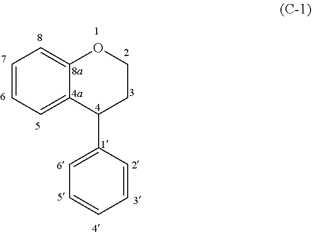 [SMILES: c1ccc(C2CCOc3ccccc32)cc1]

In formula (C-1), the atoms of the core structure have numbered in accordance with the IUPAC numbering scheme for this sub-class of compounds.

For the avoidance of doubt, formula (C-1) represents the core structure or skeleton of a 4-flavonoid. Rings A to C of the core structure may include substituents. Ring C may include a carbon-carbon double bond (in addition to the unsaturation arising from the fused phenyl group, which is ring A) and/or a carbonyl group.

In general, the term “4-flavonoid” or “neoflavonoid” includes neoflavans and neoflavones.

When ring C in formula (C-1) does not include a carbon-carbon double or a carbonyl group, then formula (C-1) represents a neoflavan. The term “neoflavan” refers to a compound with a 4-phenyl-3,4-dihydro-2H-1-benzopyran skeleton.

When ring C in formula (C-1) includes a carbonyl group, such as, for example, when the compound is derived from a 4-phenyl-2H-1-benzopyran-2-one (4-phenyl-2H-chromen-2-one) skeleton, then the 4-flavonoid is a neoflavanone (e.g. as represented by formula (C-2)).

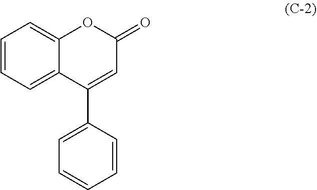 [SMILES: O=c1cc(-c2ccccc2)c2ccccc2o1]

The term “ellagic acid” as used herein refers to the following compound.

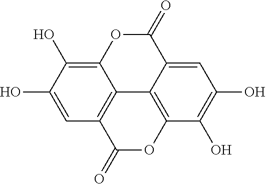 [SMILES: O=c1oc2c(O)c(O)cc3c(=O)oc4c(O)c(O)cc1c4c23]

Ellagic acid is a polyphenol, which is obtained or is obtainable from the natural extracts of many fruits, vegetables and nuts, such as blackberries, raspberries, strawberries, cranberries, walnuts, pecans, pomegranates and wolfberries.

The term “glucogallin” as used herein refers to the following compound.

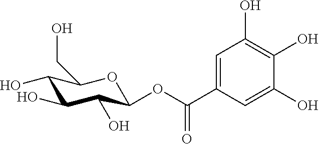

Glucogallin is typically present in a natural extract of saberry. Thus, glucogallin may be obtained or is obtainable from saberry extract.

The term “saberry extract” as used herein refers to an extract of fruits of emblica officinalis (synonymous with phyllanthus emblica), which is more commonly known as amla berry or Indian gooseberry.

As used herein by itself or in conjunction with another term or terms, “substituted” indicates that a hydrogen atom on a molecule has been replaced with a different atom or group of atoms. The atom or group of atoms replacing the hydrogen atom is a “substituent.” It should be understood that the terms “substituent”, “substituents”, “moiety”, “moieties”, “group”, or “groups” refer to substituent(s).

The various hydrocarbon-containing moieties provided herein may be described using a prefix designating the minimum and maximum number of carbon atoms in the moiety, namely “Cₐ-C_{b}”. For example, Cₐ-C_{b} alkyl indicates an alkyl moiety having the integer “a” to the integer “b” number of carbon atoms, inclusive.

As used herein, the term “alkyl”, whether used alone or in conjunction with another term, refers to a branched or unbranched saturated hydrocarbon chain, unless specified otherwise. An alkyl group is typically unsubstituted, unless indicated otherwise. An alkyl group may have 1 to 6 carbon atoms, such as 1 to 4 carbon atoms. Representative examples include, but are not limited to, methyl, ethyl, n-propyl, i-propyl, n-butyl, i-butyl, s-butyl, t-butyl, n-pentyl, n-hexyl.

As used herein, the term “alkoxy” refers to an alkyl-oxy group, wherein the alkyl group is as previously defined. An alkoxy group is typically unsubstituted, unless specified otherwise. Representative examples include —OMe, —OEt, —OⁿPr, —OⁱPr.

Amounts of a material, such as a compound, composition or an ingredient, are typically defined in terms of the wt % of the composition.

The term “about” when used herein in conjunction with a measurable value encompasses reasonable variations of the value, for instance, to allow for experimental error in the measurement of said value.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

For the avoidance of doubt, the expression “consists essentially of” as used herein limits the scope of a feature to include the specified materials, and any other materials or steps that do not materially affect the basic and novel characteristics of that feature, such as, for example, minor impurities. The expression “consists essentially of” embraces the expression “consisting of”.

### Antiperspirant Agent

The invention relates to the use of products that are naturally derived or obtainable as antiperspirant agents.

The antiperspirant agent is for reducing or preventing perspiration. Thus, the antiperspirant agent is an active ingredient for reducing or preventing perspiration in the antiperspirant composition of the invention.

The antiperspirant agent is for external use (e.g. on the human body).

The antiperspirant composition or the antiperspirant agent is for application to a surface of a body, particularly the skin.

The antiperspirant agent comprises, a flavonoid, a phenylpropanoid compound comprising a carboxylic acid (—COOH) group or a mixture thereof (e.g. a flavonoid and a phenylpropanoid compound comprising a carboxylic acid group). The antiperspirant agent may consist essentially of one or more flavonoids, phenylpropanoid compound comprising a carboxylic acid or a mixture thereof (e.g. one or more flavonoids and one or more phenylpropanoid compounds, each comprising a carboxylic acid group).

Flavonoids are a class of polyphenolic compounds, which are typically secondary metabolites found in plants.

For all aspects of the invention, the antiperspirant agent will now be further described by way of the following numbered paragraphs:
1. The antiperspirant agent comprises, or consists essentially of, a flavonoid, an optionally substituted benzenepropenoic acid, an ester of gallic acid, ellagic acid or a mixture of two or more thereof.
2. The antiperspirant agent in paragraph 1 comprising an optionally substituted benzenepropenoic acid, preferably a trans-benzenepropenoic acid.
3. The antiperspirant agent in paragraph 2, wherein the optionally substituted benzenepropenoic acid is represented by formula (I):

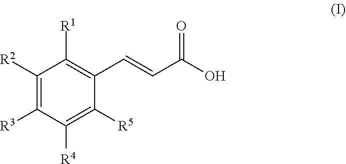

wherein each of R¹ to R⁵ is independently selected from H, hydroxy and C₁-C₆ alkoxy.

The hydroxy (—OH) and the C₁-C₆ alkoxy groups are the optional substituents mentioned in paragraphs 1 and 2.

Benzenepropenoic acids can show excellent antiperspirant activity, as illustrated by the results for trans-cinnamic acid.
4. The antiperspirant agent in paragraph 3, wherein the C₁-C₆ alkoxy is either ethoxy or methoxy.
5. The antiperspirant agent in paragraph 4, wherein the C₁-C₆ alkoxy is methoxy.
6. The antiperspirant agent in any one of paragraphs 3 to 5, wherein at least three of R¹ to R⁵ are hydrogen.
7. The antiperspirant agent in any one of paragraphs 3 to 6, wherein two adjacent groups of R¹ to R⁵ is each independently selected from hydroxy and C₁-C₆ alkoxy (or the C₁-C₆ alkoxy defined in paragraphs 4 or 5).

For example, (i) R¹ and R² or (ii) R² and R³ is each independently selected from hydroxy and C₁-C₆ alkoxy (or the C₁-C₆ alkoxy defined in paragraphs 4 or 5).
8. The antiperspirant agent in any one of paragraphs 3 to 7, wherein R¹, R⁴ and R⁵ are hydrogen, and each of R² and R³ is independently selected from hydroxy and C₁-C₆ alkoxy (or the C₁-C₆ alkoxy defined in paragraphs 4 or 5).
9. The antiperspirant agent in any one of paragraphs 1 to 3, wherein the optionally substituted benzenepropenoic acid is selected from cinnamic acid, o-coumaric acid, m-coumaric acid, p-coumaric acid, caffeic acid, ferulic acid, 5-hydroxyferulic acid and sinapic acid.
10. The antiperspirant agent in paragraph 9, wherein the optionally substituted benzenepropenoic acid is caffeic acid.
11. The antiperspirant agent in paragraph 9, wherein the optionally substituted benzenepropenoic acid is trans-cinnamic acid.

Trans-cinnamic acid may be found in some cinnamon extracts.
12. The antiperspirant agent in paragraph 1, which consists essentially of the optionally substituted benzenepropenoic acid defined in any of paragraphs 2 to 11.
13. The antiperspirant agent in paragraph 1 comprising an optionally substituted benzenepropenoic acid defined in any of paragraphs 2 to 11 and ellagic acid. The antiperspirant agent may be a combination or a mixture of the optionally substituted benzenepropenoic acid and ellagic acid.
14. The antiperspirant agent in paragraph 1 comprising ellagic acid.
15. The antiperspirant agent in paragraph 1, which consists essentially of ellagic acid.
16. The antiperspirant agent in paragraph 1 comprising an ester of gallic acid.
17. The antiperspirant agent in paragraph 1 or paragraph 16, wherein the ester of gallic acid is selected from a flavonoid ester of gallic acid and a sugar ester of gallic acid.
18. The antiperspirant agent in paragraph 17, wherein the ester of gallic acid is a sugar ester of gallic acid.
19. The antiperspirant agent in paragraph 17 or paragraph 18, wherein the sugar ester of gallic acid is glucogallin (e.g. β-glucogallin).
20. The antiperspirant agent in paragraph 17, wherein the ester of gallic acid is a flavonoid ester of gallic acid.
21. The antiperspirant agent in paragraph 17 or paragraph 20, wherein the flavonoid ester of gallic acid is a flavan-3-ol ester of gallic acid. The flavan-3-ol ester of gallic acid typically comprises following structural moiety:

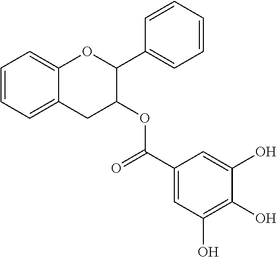 [SMILES: O=C(OC1Cc2ccccc2OC1c1ccccc1)c1cc(O)c(O)c(O)c1]

The term “moiety” is used because the above structure may contain one or more substituents on the 2-flavonoid skeleton or core structure.
22. The antiperspirant agent in paragraph 21, wherein the flavan-3-ol ester of gallic acid is selected from catechin gallate, epicatechin gallate, epigallocatechin gallate and a mixture of two or more thereof.
23. The antiperspirant agent in paragraph 21, wherein the flavan-3-ol ester of gallic acid is epigallocatechin gallate.

The green tea extract used in the examples contained at least 30 wt % of epigallocatechin gallate.
24. The antiperspirant agent in paragraph 21, wherein the flavan-3-ol ester of gallic acid is selected from catechin gallate, epicatechin gallate and a mixture thereof.

The cinnamon extract used in the examples contained a mixture of catechin gallate and epicatechin gallate.
25. The antiperspirant agent in paragraph 1, which consists essentially of the ester of gallic acid defined in any of paragraphs 16 to 24.
26. The antiperspirant agent in paragraph 1 comprising an optionally substituted benzenepropenoic acid defined in any of paragraphs 2 to 11, such as trans-cinnamic acid, and an ester of gallic acid defined in any of paragraphs 16 to 24. The antiperspirant agent may be a combination or a mixture of the optionally substituted benzenepropenoic acid and the ester of gallic acid.
27. The antiperspirant agent in paragraph 1 comprising an ester of gallic acid defined in any of paragraphs 16 to 24 and ellagic acid. The antiperspirant agent may be a combination or a mixture of the ester of gallic acid and ellagic acid.
28. The antiperspirant agent in paragraph 1 comprising a flavonoid.
29. The antiperspirant agent in paragraph 1 or paragraph 28, wherein the flavonoid is a 2-flavonoid, a 3-flavonoid or a 4-flavonoid.
30. The antiperspirant agent in paragraph 1, 28 or 29, wherein the flavonoid has the general structure:

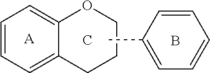

31. The antiperspirant agent in paragraph 1 or 28 to 30, wherein the flavonoid is a 2-flavonoid, a 3-flavonoid or a mixture of two or more thereof.
32. The antiperspirant agent in paragraph 31, wherein the flavonoid is a 2-flavonoid or a mixture of 2-flavonoids.
33. The antiperspirant agent in paragraph 31 or paragraph 32, wherein the or each flavonoid has at least one substituent on ring A, wherein each substituent is independently selected from hydroxy (—OH) and C₁₋₆ alkoxy.
34. The antiperspirant agent in paragraph 33, wherein the or each flavonoid has two or more substituents on ring A, preferably two (e.g. only two) substituents on ring A, wherein each substituent is independently selected from hydroxy and C₁₋₆ alkoxy.
35. The antiperspirant agent in paragraph 33 or paragraph 34, wherein the C₁-C₆ alkoxy is either ethoxy or methoxy.
36. The antiperspirant agent in paragraph 35, wherein the C₁-C₆ alkoxy is methoxy.
37. The antiperspirant agent in paragraph 33, wherein each substituent is hydroxy.
38. The antiperspirant agent in paragraph 34, wherein the two or more substituents on ring A, preferably two (e.g. only two) substituents on ring A, are hydroxy (—OH).
39. The antiperspirant agent in any one of paragraphs 33 to 38, wherein one substituent on ring A is at the 5-position.
40. The antiperspirant agent in any one of paragraphs 33 to 38, wherein one substituent on ring A is at the 7-position.
41. The antiperspirant agent in any one of paragraphs 34 to 38, wherein the two or more substituents on ring A, preferably two (e.g. only two) substituents on ring A, are at least at the 5-position and the 7-position of ring A.
42. The antiperspirant agent in any one of paragraphs 31 to 41, wherein the or each flavonoid has at least one substituent on ring B, wherein each substituent is independently selected from hydroxy (—OH) and C₁₋₆ alkoxy.
43. The antiperspirant agent in paragraph 42, wherein the or each flavonoid has two or more substituents on ring B, wherein each substituent is independently selected from hydroxy and C₁₋₆ alkoxy.
44. The antiperspirant agent in paragraph 43, wherein the or each flavonoid has three or more substituents on ring B, wherein each substituent is independently selected from hydroxy and C₁₋₆ alkoxy.
45. The antiperspirant agent in any of paragraphs 42 to 44, wherein the C₁-C₆ alkoxy is either ethoxy or methoxy.
46. The antiperspirant agent in paragraph 45, wherein the C₁-C₆ alkoxy is methoxy.
47. The antiperspirant agent in paragraph 42, wherein each substituent is hydroxy.
48. The antiperspirant agent in paragraph 42, wherein the at least one substituent on ring B is hydroxy (—OH).
49. The antiperspirant agent in paragraph 43, wherein the two or more substituents on ring B are hydroxy (—OH).
50. The antiperspirant agent in paragraph 44, wherein the three or more substituents on ring B are hydroxy (—OH).
51. The antiperspirant agent in any of paragraphs 42 or 45 to 48, wherein the at least one substituent is at the 3′-position, the 4′-position or the 5 position of ring B, preferably the 4′-position of ring B.
52. The antiperspirant agent in any of paragraphs 43 or 45 to 48, where the two or more substituents are at the 4′-position and the 3′-position of ring B.
53. The antiperspirant agent in any of paragraphs 44 to 48, wherein the three or more substituents are at the 3′-position, the 4′-position and the 5-position of ring B.
54. The antiperspirant agent in any of paragraphs 31 to 53, wherein the 2-flavonoid is a flavanol or an ester of a flavanol, or the mixture of 2-flavonoids defined in paragraph 32 includes a flavanol and/or an ester of a flavanol.
55. The antiperspirant agent in paragraph 54, wherein the or each flavanol is a flavan-3-ol.
57. The antiperspirant agent in paragraph 54 or paragraph 55, wherein the 2-flavonoid is afzelechin, epiafzelechin, catechin, epicatechin, gallocatechin, epigallocatechin or a combination of two or more thereof, preferably the 2-flavonoid is catechin.

Green tea extract, such as the green tea extract used in the examples, contains at least 40 to 70 wt % of catechin.
58. The antiperspirant agent in paragraph 57, wherein the 2-flavonoid is a combination of catechin, epicatechin, gallocatechin and epigallocatechin.

Cinnamon extract, such as the cinnamon extract used in the examples, contains catechin, epicatechin, gallocatechin and epigallocatechin as its main ingredients.
59. The antiperspirant agent in any of paragraphs 54 to 58, wherein the or each ester of a flavanol is an ester of a flavan-3-ol.
60. The antiperspirant agent in paragraph 59, wherein the ester of a flavan-3-ol is not a flavan-4-one. In other words, the flavan-3-ol does not have a carbonyl group at the 4-position on ring C.
61. The antiperspirant agent in paragraph 59 or paragraph 60, wherein the ester of a flavan-3-ol is a gallate ester of a flavan-3-ol, such as catechin gallate, epicatechin gallate, epigallocatechin gallate.
62. The antiperspirant agent in any of paragraphs 31 to 61, wherein the 2-flavonoid is a flavanone, or the mixture of 2-flavonoids defined in paragraph 32 or any of paragraphs 33 to 61 includes a flavanone.
63. The antiperspirant agent in paragraph 31, wherein the flavanone is a flavan-4-one.
64. The antiperspirant agent in paragraph 31, wherein the flavan-4-one has at least one substituent at the 5-position or the 7-position of ring A, wherein the at least one substituent is C₁₋₆ alkoxy.
65. The antiperspirant agent in paragraph 64, wherein the C₁-C₆ alkoxy is either ethoxy or methoxy.
66. The antiperspirant agent in paragraph 65, wherein the C₁-C₆ alkoxy is methoxy.
67. The antiperspirant agent in any of paragraphs 63 to 66, wherein the flavan-4-one is selected from rutin, 5,7-dimethoxyflavan-4-one, 5-hydroxy-7-methoxyflavan-4-one and a mixture of two or more thereof.
68. The antiperspirant agent in paragraph 67, wherein the flavan-4-one is rutin.

Cinnamon extract, such as used in the examples, contains rutin.
69. The antiperspirant agent in paragraph 67, wherein the flavan-4-one is 5,7-dimethoxyflavan-4-one.

Black ginger extract, such as used in the examples, contains 5,7-dimethoxyflavan-4-one.
70. The antiperspirant agent in paragraph 1, which consists essentially of a flavonoid as defined in any of paragraphs 30 to 69.
71. The antiperspirant agent in paragraph 1, comprising a flavonoid defined in any of paragraphs 30 to 69 and an optionally substituted benzenepropenoic acid defined in any of paragraphs 2 to 11, such as trans-cinnamic acid. The antiperspirant agent may be a combination or a mixture of the flavonoid and the optionally substituted benezepropenoic acid.

Flavonoids are often coloured, whereas the optionally substituted benzenepropenoic acid is usually a white solid. The inclusion of the optionally substituted benzenepropenoic acid may dilute the colour of the flavonoid. It may also improve the odour characteristics of the resulting product.
72. The antiperspirant agent in paragraph 1, comprising a flavonoid defined in any of paragraphs 30 to 69 and the ester of gallic acid defined in any of paragraphs 16 to 24. The antiperspirant agent may be a combination or a mixture of the flavonoid and the ester of gallic acid.
73. The antiperspirant agent in paragraph 1, comprising a flavonoid defined in any of paragraphs 30 to 69 and ellagic acid. The antiperspirant agent may be a combination or a mixture of the flavonoid and the gallic acid.
74. The antiperspirant agent in paragraph 1, comprising catechin and epigallocatechin gallate.
75. The antiperspirant agent in paragraph 74, comprising an amount of at least 40 wt % of catechin (e.g. by weight of the antiperspirant agent).
76. The antiperspirant agent in paragraph 75, wherein the amount of catechin is at least 75 wt % (e.g. of the antiperspirant agent).
77. The antiperspirant agent in paragraph 74 or paragraph 75, comprising an amount of at least 15 wt % epigallocatechin gallate (e.g. by weight of the antiperspirant agent).
78. The antiperspirant agent in paragraph 77, wherein the amount of epigallocatechin gallate is at 30 wt % (e.g. by weight of the antiperspirant agent).
79. The antiperspirant agent in paragraph 1, comprising catechin, epicatechin, afzelechin, epiafzelechin, catechin gallate, epicatechin gallate, gallocatechin, epigallocatechin and rutin.
80. The antiperspirant agent in any of paragraphs 1 to 79 obtained or obtainable from a natural extract.
81. The antiperspirant agent in paragraph 80, wherein the natural extract is selected from cinnamon extract, black ginger extract, green tea extract, saberry extract and a mixture of two or more thereof.
82. The antiperspirant agent in any of paragraphs 1 to 81, which does not include an aluminum chlorohydrate or aluminum zirconium tetrachlorohydrex gly.
83. The antiperspirant agent in any of paragraphs 1 to 81, which does not include an aluminum-containing antiperspirant agent, preferably does not include aluminum.
84. The antiperspirant agent in paragraph 83, which does not include zirconium.

The antiperspirant agent in any one of paragraphs 1 to 84 above) may be present in the antiperspirant composition in the form of the natural extract, such as in the second aspect of the invention.

For all aspects of the invention, the natural extract will now be further described by way of the following numbered paragraphs:
85. The natural extract may be selected from cinnamon extract, black ginger extract, green tea extract, saberry extract and a mixture of two or more thereof.
86. The natural extract in paragraph 85, which is black ginger extract.
87. The natural extract in paragraph 85 or paragraph 86, wherein the black ginger extract comprises a flavan-4-one, preferably a plurality of flavan-4-ones.
88. The natural extract in paragraph 87, wherein the flavan-4-one, or at least one of the plurality of flavan-4-ones, is 5,7-dimethoxyflavan-4-one.
89. The natural extract in paragraph 87 or paragraph 88, wherein the flavan-4-one, or at least one of the plurality of flavan-4-ones, is 5-hydroxy-7-methoxyflavan-4-one.
90. The natural extract in paragraph 85, which is cinnamon extract.
91. The natural extract in paragraph 85 or paragraph 90, wherein the cinnamon extract comprises one or more 2-flavonoids.
92. The natural extract in paragraph 91, wherein at least one of the one or more 2-flavonoids is a flavanol, preferably a flavan-3-ol.
93. The natural extract in paragraph 92, wherein the flavanol or the flavan-3-ol is afzelechin, epiafzelechin, catechin, epicatechin, gallocatechin, epigallocatechin or a combination of two or more thereof.
94. The natural extract in any of paragraphs 91 to 93, wherein at least one of the one or more 2-flavonoids is a flavanone, preferably a flavan-4-one.
95. The natural extract in paragraph 94, wherein the flavanone or the flavan-4-one is rutin.
96. The natural extract in any of paragraphs 85 or 91 to 95, wherein the cinnamon extract comprises cinnamic acid, preferably trans-cinnamic acid.
97. The natural extract in paragraph 85, which is saberry extract.
98 The natural extract in paragraph 85 or paragraph 97, wherein the saberry extract comprises glucogallin (e.g. β-glucogallin).
99. The natural extract in paragraph 85, which is green tea extract.
100. The natural extract in paragraph 85 or paragraph 99, wherein the green tea extract comprises catechin, epigallocatechin gallate or a combination thereof, preferably a combination of catechin and epigallocatechin gallate.

Without wishing to be bound by theory, the antiperspirant agents of the invention, whether present as individual compounds or in the form of a natural extract, may plug the pores of sweat glands by causing protein aggregation and/or by binding to specific receptors in the wall of the sweat duct.

It has surprisingly been found that flavonoids and cinnamic acid have an antiperspirant effect. It is believed that these compounds cause coagulation or agglomeration of proteins.

### Antiperspirant Composition

The antiperspirant composition of the invention comprises an antiperspirant agent as defined in the headed section above, such as in any of paragraphs 1 to 84. The antiperspirant agent may be present as a natural extract as defined above, particularly in paragraphs 85 to 100. The antiperspirant agent may be obtained or obtainable from the natural extract.

Typically, the antiperspirant agent as defined above is the sole antiperspirant agent in the antiperspirant composition.

When the antiperspirant composition comprises a natural extract as defined above, then it is preferred that the natural extract is the sole antiperspirant agent.

In general, the antiperspirant composition does not comprise aluminum chlorohydrate or aluminum zirconium tetrachlorohydrex gly. It is preferred that the antiperspirant composition does not comprise an aluminum-containing antiperspirant agent, more preferably the antiperspirant composition does not comprise aluminum and optionally zirconium.

The antiperspirant composition comprises an effective amount of the antiperspirant agent or the natural extract. The effective amount is an amount that will reduce or prevent perspiration.

The antiperspirant composition typically comprises a total amount of from about 0.1 to about 25.0 wt % of the antiperspirant agent, preferably from about 0.5 to about 15.0 wt %, more preferably from about 1.0 to about 10.0 wt %, such as from about 1.0 to about 5.0 wt % of the antiperspirant agent.

The antiperspirant composition preferably comprises a total amount of about 0.1 to about 4.0 wt % of the antiperspirant agent, such as about 0.5 to about 4.0 wt %, more preferably from about 1.0 to 4.0 wt %, such as about 1.0 to about 3.5 wt % of the antiperspirant agent.

The antiperspirant composition typically comprises a total amount of from about 5.0 to about 40.0 wt % of the natural extract, preferably from about 10.0 to about 30.0 wt %, more preferably from about 15.0 to about 25.0 wt % of the natural extract.

The amount of natural extract that is included in the antiperspirant composition will depend on the composition of the extract, particularly the type and amount of ingredients that are present in the natural extract.

In general, the antiperspirant composition is a cosmetic antiperspirant composition, particularly a cosmetic antiperspirant composition for personal care.

The antiperspirant composition is typically for topical use. Thus, the antiperspirant composition may be a topical antiperspirant composition.

The antiperspirant composition is for topical application to skin, preferably a surface of the skin. The antiperspirant composition may be formulated for a specific mode of topical application.

Typically, the antiperspirant composition comprises a topically acceptable carrier. The topically acceptable carrier will depend on the type of formulation and its mode of topical application.

The topically acceptable carrier may be water, an alcohol, an ether, an oil or a wax.

The topically acceptable carrier may be water or water and oil, such as when the antiperspirant composition is in the form of a gel, a paste, a liquid, a lotion or a foam.

The topically acceptable carrier may be an alcohol. The alcohol may be a mono-alcohol or a polyhydric alcohol (e.g. an alcoholic compound having two or more hydroxy groups).

When the alcohol is a mono-alcohol, then then mono-alcohol may be ethanol or a fatty alcohol. The fatty alcohol may have a straight chain length of 6 to 18 carbon atoms. It is preferred that the fatty alcohol is stearyl alcohol.

When the alcohol is a polyhydric alcohol, then the polyhydric alcohol may be a polymeric alcohol or a monomeric alcohol. The polymeric alcohol may be a polyalkylene glycol, such as PEG or PPG.). The monomeric alcohol may be ethylene glycol, propylene glycol, glycerol or a sugar, such as glucose, sorbitol or xylitol.

The topically acceptable carrier may be an ether. The ether may be PPG-14 butyl ether.

The topically acceptable carrier may be an oil. The oil may be hydrogenated castor oil.

The antiperspirant composition may be provided as, or included in, a deodorant, such as a body deodorant for personal hygiene purposes.

The antiperspirant composition may be in the form of a cream, a gel, a paste, a liquid, a lotion, a foam, a scrub or a powder.

Typically, the antiperspirant composition is a leave-on composition. A leave on composition may be applied to skin, or a surface of the skin, and left on for a period of time (e.g. from 1 minute to 24 hours), after which it may be wiped or rinsed off with water, usually during the regular course of washing.

When the antiperspirant composition is in the form of a liquid, then the liquid may be a water-in-oil emulsion or an oil-in-water emulsion.

In general, when the antiperspirant composition is in the form of a liquid, then the liquid may be a sprayable liquid.

The antiperspirant composition may be for delivery as an aerosol. The topically acceptable carrier may comprise a propellant.

The antiperspirant composition may comprise a lubricant. The lubricant may, for example, be a cyclomethicone, a dimethicone, a phenyltrimethicone or an ethyl methicone. It is preferred that the lubricant is a cyclomethicone and/or phenyltrimethicone.

The antiperspirant composition of the invention may further comprise a natural oil. The natural oil may be included for skin care benefits.

Typically, the natural oil comprises an unsaturated fat, such as a polyunsaturated fat (e.g. a triglyceride derived from linoleic acid) and/or a monounsaturated fat (e.g. a triglyceride derived from oleic acid).

The antiperspirant composition may further comprise an antioxidant ingredient. An antioxidant ingredient may be included to chemically stabilise the antiperspirant or any other natural ingredient that may be present, such a natural oil, particularly a natural oil comprising a polyunsaturated fat and/or a monounsaturated fat.

The antioxidant ingredient may be selected from ascorbic acid, butyl hydroxy toluene, pentaerythrityl tetra-di-t-butyl hydroxyhydrocinnamate and tris-(tetramethylhydroxypiperidinol) citrate.

The antiperspirant composition may include a fragrance.

The antiperspirant composition may include a thickener. The thickener may be fumed silica or a cellulose ether, such as hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose or xanthan gum. It is preferred that the thickener is fumed silica.

The antiperspirant composition may include a surfactant. The surfactant may be an anionic surfactant, a cationic surfactant, a non-ionic surfactant or an amphoteric surfactant.

The antiperspirant composition may include a personal care ingredient. The personal care ingredient may be a skin care agent, such as an emollient, a humectant, a skin barrier promoter or a skin appearance modifier.

The antiperspirant composition may comprise a wide range of other optional components, such as a binder, a biological additive, a buffering agent, a colorant, a fragrance, a conditioner, an exfoliating agent, a pH adjuster, a preservative, a skin soothing agent or a skin healing agent.

The antiperspirant composition can be manufactured by conventional methods known in the art.

### Methods and Uses

The method or uses of the invention are non-therapeutic.

The antiperspirant composition of the invention is for use as a cosmetic, particularly for personal care.

The antiperspirant composition or the antiperspirant agent is applied to skin, preferably topically applied to skin.

The antiperspirant composition or the antiperspirant agent is applied to a surface of the skin.

In general, the skin is of a mammal, preferably a human.

Typically, the antiperspirant composition or the antiperspirant agent is applied to an area of skin where perspiration occurs, preferably an area of the skin where there are sweat glands, such as apocrine sweat glands or eccrine sweat glands, preferably apocrine sweat glands.

The area of skin may be on a thigh (e.g. inner thigh), the groin, the back, the upper lip, an armpit, the forehead, the knee (e.g. the back of the knee), the neck (e.g. back of the neck), the buttock, the chest, the scalp or the hand. It is preferred that the area of skin is an armpit.

The antiperspirant composition or the antiperspirant agent may be applied by a direct body contact applicator, such as a roll-on lotion or a solid stick product.

Alternatively, the antiperspirant composition or the antiperspirant agent may be applied by a non-contact applicator, such as by spraying onto the skin. The non-contact applicator may be an aerosol spray, a pump spray or a squeeze spray.

### EXAMPLES

The invention will now be illustrated by the following non-limiting examples.

### Examples 1 to 8

Formulation examples 1 to 8 shown in Table 1 were prepared by conventional methods.

TABLE 1

| | Formulation (F) No. | | | | | | | |
|---|---|---|---|---|---|---|---|---|
| Ingredient | 1 | 2 | 3 | 4 | 5 | 6 | 7 | 8 |
| Natural extract | E1 | E2 | E2 + E3 | E4 | E5 | E3 + E5 | E6 | E3 |
| Cyclomethicone | ✓ | ✓ | ✓ | ✓ | ✓ | ✓ | ✓ | ✓ |
| Aluminum starch | ✓ | ✓ | ✓ | ✓ | ✓ | ✓ | ✓ | ✓ |
| succinate | | | | | | | | |
| Phenyltrimethicone | ✓ | ✓ | ✓ | ✓ | ✓ | ✓ | ✓ | ✓ |
| PPG-14 butyl ether | ✓ | ✓ | ✓ | ✓ | ✓ | ✓ | ✓ | ✓ |
| Fumed silica | ✓ | ✓ | ✓ | ✓ | ✓ | ✓ | ✓ | ✓ |
| Hydrogenated | ✓ | ✓ | ✓ | ✓ | ✓ | ✓ | ✓ | ✓ |
| castor oil | | | | | | | | |
| Stearyl alcohol | ✓ | ✓ | ✓ | ✓ | ✓ | ✓ | ✓ | ✓ |
| 3-phenoxyethanol- | ✓ | ✓ | ✓ | ✓ | ✓ | ✓ | ✓ | ✓ |
| 1,2-propanediol | | | | | | | | |

In Table 1 above, E1 is thai black ginger extract; E2 is cinnamon extract; E3 is saberry; E4 is ellagic acid; E5 is green tea extract; and E6: trans-cinnamic acid. The extracts were obtained from Sabinsa Corporation. Each natural extract was included in an amount in each formulation to provide the same amount of antiperspirant agent across the formulations.

### COMPARATIVE EXAMPLES

The formulations shown in Table 2 were prepared to provide a comparison to the formulations in Table 1. No natural extracts were included in these formulations.

TABLE 2

Comparative Formulation (CF) No.

| Ingredient | 1 | 2 |
|---|---|---|
| Natural extract | — | — |
| Antiperspirant | AP1 | AP2 |
| Cyclomethicone | ✓ | ✓ |
| Aluminum starch | ✓ | ✓ |
| octenylsuccinate | | |
| Phenyltrimethicone | ✓ | ✓ |
| PPG-14 butyl ether | ✓ | ✓ |
| Fumed silica | ✓ | ✓ |
| Hydrogenated castor oil | ✓ | ✓ |
| Stearyl alcohol | ✓ | ✓ |

In Table 2 above, AP1 is aluminum chlorohydrate and AP2 is aluminum zirconium tetrachlorohydrate glycine (AZG).

### Roll-On Antiperspirant Product

The method of manufacturing a roll-on product is set out below.
1. Mix cyclomethicone, phenyltrimethicone, stearyl alcohol, PPG-14 butyl ether and the hydrogenated castor oil.
2. Heat to 82° C. to melt the solids.
3. While maintaining a temperature of 82° C., slowly add silica using a high-speed disk type dispenser.
4. Once all silica is added, increase mixing speed so that peripheral velocity is almost 4000 ft/minute or higher for satisfactory dispersion.
5. Add starch followed by the Natural AP powder.
6. It is important to disperse each component uniformly before adding the next.
7. Once all components are added, cool the mixture to 55° C.

### In Vivo Test Procedure

### Study Design

The formulation examples were tested in a standard hot room assessment clinical study. The study panellists underwent thermal stress after application of the antiperspirant compositions. The sweat reduction was determined by gravimetric collection of the sweat.

Subjects: 40 female panellists.

Conditions: 17-day washout period in which the panellists did not use any antiperspirants.

Test Design: Subjects underwent 5-day application period where each was given 400 mg+50 mg of one of the formulation examples. After the 5th day, sweat collection was conducted after the panellists entered the hot room (100° F.±2° F. and 35%±5% relative humidity) for 80 minutes to induce sweating.

Analysis of Data: The data were transformed to their natural logarithms and the averages were analyzed according to the method described by Murphy and Levine (Reference 2). The analysis of covariance model included factors for sequence, subjects within a sequence, test articles, avilla and baseline as the covariate.

For each formulation, the % Change in sweat reduction was determined. The clinical study data showed that all of the formulations were effective at reducing sweat, with formulations F1 to F8 showing a sweat reduction greater than 30% when compared to baseline. A sweat reduction of at least 20% is considered to be suitable for commercial use as an antiperspirant agent.

The comparative formulations showed a sweat reduction greater than 50%. A few of the prototypes (e.g. formulations F7, F8 and F5) showed sweat efficacy results that are parity results when compared to the products with aluminum and aluminum zirconium.

### REFERENCES

The following documents are referenced in the description.
1. A. P. Rauter et al.: “Nomenclature of flavonoids (IUPAC Recommendations 2017)”; Pure Appl. Chem., 90(9), (2018), 1429-1486.
2. T. D. Murphy & M. J. Levine: “Analysis of Antiperspirant Efficacy Test Results”; Journal of the Society of Cosmetic Chemists, 42, (1991), 167-197.

The entire disclosure of each document cited herein is hereby incorporated herein by reference. All references cited in this disclosure are incorporated by reference to the same extent as if each reference had been incorporated by reference in its entirety individually.

The citation and incorporation of patent documents herein is done for convenience only and does not reflect any view of the validity, patentability, and/or enforceability of such patent documents.

A number of embodiments of the disclosure have been described. Nevertheless, it will be understood that various modifications may be made without departing from the spirit and scope of the present disclosure. Accordingly, other embodiments are within the scope of the following claims.

All headings and sub-headings are used herein for convenience only and should not be construed as limiting the invention in any way.

The use of any and all examples, or exemplary language (e.g. “such as”) provided herein, is intended merely to better illuminate the invention and does not pose a limitation on the scope of the invention unless otherwise paragraphed. No language in the specification should be construed as indicating any non-paragraphed element as essential to the practice of the invention.

1. An antiperspirant composition comprising an antiperspirant agent, wherein the antiperspirant agent comprises an optionally substituted benzenepropenoic acid, a flavonoid, an ester of gallic acid, ellagic acid or a mixture of two or more thereof.

2-3. (canceled)

4. The antiperspirant composition according to claim 1, wherein the optionally substituted benzenepropenoic acid is represented by formula (I): 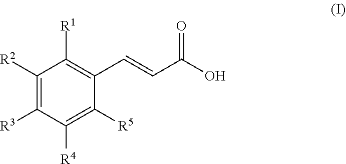
wherein each of R¹ to R⁵ is independently selected from H, hydroxy and C₁-C₆ alkoxy.

5-7. (canceled)

8. The antiperspirant composition according to claim 1, wherein the optionally substituted benzenepropenoic acid is selected from cinnamic acid, o-coumaric acid, m-coumaric acid, p-coumaric acid, caffeic acid, ferulic acid, 5-hydroxyferulic acid and sinapic acid.

9. (canceled)

10. The antiperspirant composition according to claim 1, wherein the antiperspirant agent comprises ellagic acid.

11. The antiperspirant composition according to claim 1, wherein the antiperspirant agent comprises an ester of gallic acid.

12-16. (canceled)

17. The antiperspirant composition according to claim 1, wherein the flavonoid has at least one substituent on ring A, wherein each substituent is independently selected from hydroxy (—OH) and C₁₋₆ alkoxy.

18. The antiperspirant composition according to claim 17, wherein the flavonoid has two or more substituents on ring A, wherein each substituent is independently selected from hydroxy and C₁₋₆ alkoxy.

19-21. (canceled)

22. The antiperspirant composition according to claim 1, wherein the flavonoid is a 2-flavonoid.

23-26. (canceled)

27. The antiperspirant composition according to claim 22, wherein the 2-flavonoid is a gallate ester of a flavan-3-ol, preferably catechin gallate, epicatechin gallate, epigallocatechin gallate.

28-31. (canceled)

32. The antiperspirant composition according to claim 1, wherein the antiperspirant agent is obtained or obtainable from a natural extract, preferably a natural extract selected from cinnamon extract, black ginger extract, green tea extract, saberry extract and a mixture of two or more thereof.

33. The antiperspirant composition according to claim 1, comprising a total amount of from about 0.1 to about 25.0 wt % of the antiperspirant agent.

34. An antiperspirant composition comprising a natural extract selected from cinnamon extract, black ginger extract, green tea extract, saberry extract and a mixture of two or more thereof.

35. (canceled)

36. The antiperspirant composition according to claim 35, wherein the black ginger extract comprises a flavan-4-one, preferably a plurality of flavan-4-ones.

37-38. (canceled)

39. The antiperspirant composition according to claim 34, wherein the cinnamon extract comprises one or more 2-flavonoids.

40-43. (canceled)

44. The antiperspirant composition according to claim 38, wherein the cinnamon extract comprises cinnamic acid, preferably trans-cinnamic acid.

45-47. (canceled)

48. The antiperspirant composition according to claim 34, wherein the green tea extract comprises catechin, epigallocatechin gallate or a combination thereof.

49. The antiperspirant composition according to claim 34, comprising a total amount of from about 5.0 to about 40.0 wt % of the natural extract.

50. The antiperspirant composition according to claim 1 further comprising a topically acceptable carrier.

51. The antiperspirant composition according to claim 1, which is in the form of a cream, a gel, a paste, a liquid, a lotion, a foam, a scrub or a powder.

52. (canceled)

53. The antiperspirant composition according to claim 1 further comprising a fragrance.

54-58. (canceled)
